(19) United States
(12) Patent Application Publication
(10) Pub. No.: US 2014/0081120 A1
(43) Pub. Date: Mar. 20, 2014

(54) SYSTEM AND METHOD FOR DETECTING TISSUE SURFACE PROPERTIES

(71) Applicant: Vanderbilt University, Nashville, TN (US)

(72) Inventors: Pietro Valdastri, Nashville, TN (US); Marco Beccani, Nashville, TN (US); Christian Di Natali, Nashville, TN (US)

(73) Assignee: Vanderbilt University, Nashville, TN (US)

(21) Appl. No.: 14/027,561

(22) Filed: Sep. 16, 2013

Related U.S. Application Data
(60) Provisional application No. 61/701,447, filed on Sep. 14, 2012.

(51) Int. Cl. A61B 5/05; A61B 5/11; A61B 5/00

(52) U.S. Cl. CPC A61B 5/05; A61B 5/4887; A61B 5/6846; A61B 5/742; A61B 5/1126
USPC 600/409

(57) ABSTRACT

A system and method of detecting and assessing a tissue surface property without a separate access port to internal anatomical structures. The system includes a first unit positioned outside the patient's body and a second unit positioned inside the patient's body. The first unit includes a magnetic field source and a force sensor and is positioned outside the patient's body in a position that enables magnetic coupling with the second unit, which is inside the patient's body. The second unit includes a magnetic field source, a processor, a sensor, a telemetry unit, a power source, and an optional actuator or other components. The resulting attractive force between the internal and external magnetic field sources can be perceived by the force sensor of the first unit. By varying the distance between the two units, the attractive force triggers a variable stress on the tissue surrounding the second unit in the direction of the magnetic field source in the first unit.

### RELATED APPLICATIONS

This application claims the benefit of U.S. Provisional Patent Application No. 61/701,447, filed on Sep. 14, 2012, the entire contents of which are incorporated herein by reference.

### BACKGROUND OF THE INVENTION

Palpation is commonly used in open surgery to manually detect tissue abnormalities. Manual palpation typically requires open surgery with large incisions, and therefore longer recovery times for the patient. During open surgery, surgeons use their hands to access the anatomy and to feel their way around sensitive anatomical structures and to correlate the actual anatomy with preoperative data. Examples where surgeons employ manual palpation include identification of underlying arteries during dissection, identification of hepatic aneurysms during liver surgery, intramedullary fixation of tibia and femur during orthopedic surgery, assistance during adenoidectomy procedures, and identification of laryngeal nerves during thyroid surgery. Additional examples includes surgeons using manual palpation to search for abnormalities such as breast masses, cancer, heart and liver enlargement, to identify active ulcers, and to localize aneurysms.

Manual palpation capabilities are unfortunately lost during minimally invasive surgery (“MIS”), which has many other advantages such as trauma reduction, improved cosmesis, shortened recovery time, and reduced hospitalization costs.

Some devices that restore palpation feedback have been proposed for MIS, but none of them have been translated to clinical application so far. One of the main reasons is that devoting one of the few abdominal access ports in a minimally invasive procedure to an instrument that tries to restore palpation has never been considered to be a wise investment for the sake of surgical outcomes. Despite progress in robotic assistance, existing MIS robotic systems do not support palpation, and they are predominantly passive “motion replicators” (i.e., the robot grippers follow direct or scaled motions of the surgeon's hands). To date, there are no algorithms that enable robots to use in-vivo sensory palpation data to actively augment the surgeon's perception of the surgical field or assist in in vivo diagnostics and in task execution. Also, from a design perspective, existing robotic MIS systems are increasingly able to restore dexterous surgical intervention capabilities typically available to surgeons during open surgery. These systems however, are limited by both physical designs and by their control algorithms. Design limitations restrict their use to trans-cutaneous access in bodily cavities by using 3-5 access ports while having a physical connection to extracorporeal actuation devices, which limit end-effector travel within the patient's body.

A wireless palpation technique would not consume port space and can be used beyond minimally invasive surgery, whenever the proposed invention can be introduced by natural orifices or tiny incisions.

### SUMMARY OF THE INVENTION

The present invention relates to systems and methods for providing clinicians with haptic feedback during minimally invasive surgery (“MIS”). In particular, the present invention relates to detecting and assessing a tissue surface property without a separate access port to internal structures.

The present invention relates to a wireless palpation device (“WPD”) which provides a novel approach to detecting underlying tissue abnormalities. By way of example, the present invention may be used in colorectal cancer detection, detection of abnormalities in the GI tract, and detection of abnormalities in abdominal organs during MIS.

The present invention includes two units: a first unit positioned outside the patient's body and a second unit positioned inside the patient's body. The first unit includes a magnetic field source and a force sensor and is positioned outside the patient's body in a position that enables magnetic coupling with the second unit, which is inside the patient's body. The second unit includes a magnetic field source and other components, and thereby the resulting attractive force between the magnetic field sources can be perceived by the force sensor of the first unit. By varying the distance between the two units, the attractive force triggers a variable stress on the tissue surrounding the second unit in the direction of the magnetic field source in the first unit.

In one embodiment, the invention provides a system for detecting a tissue property. The system includes a first or outer unit located on a first side of the tissue surface. The first unit includes a housing have a sensor and a magnetic field source. The system also includes a second or inner unit located on a second side of the tissue surface. The second unit includes a housing that supports at least one sensor, a magnetic field source, a controller, a telemetry unit, and a power source. The first unit is magnetically coupled to the second unit such that a force created therebetween triggers stress on the tissue surface. The sensor in the first unit determines a magnitude of the force between the first and second units, while the sensor in the second unit determines displacement of the tissue surface that results from the force.

In another embodiment, the invention provides a system for detecting a tissue property. The system includes a first unit positioned exterior to a patient and a second unit positioned inside of the patient near a target tissue. The first unit includes a first housing, a first sensor supported by the first housing, and a first magnetic field source supported by the housing. The second unit includes a second housing, a second sensor supported by the second housing, the second sensor positioned adjacent the target tissue, a second magnetic field source supported by the second housing, a controller supported by the second housing, a telemetry unit supported by the second housing, and a power source supported by the second housing. The first sensor is configured to detect an amount of force applied to the target tissue due to a magnetic coupling between the first unit and the second unit, and the second sensor is configured to determine a displacement of the tissue due to the magnetic coupling between the first unit and the second unit.

In a further embodiment, the invention provides a method for detecting a tissue surface contour. The method includes the steps of positioning, using a trocar, a first unit on a first side of the tissue surface at a region of interest. The first unit includes a housing that supports at least one sensor, a magnetic field source, a controller, a telemetry unit, and a power source. The method further includes providing a second unit on a second side of the tissue surface. The second unit includes a housing having a sensor and a magnetic field source. The method also includes modulating a force created by a magnetic field between the first and second units in order to determine and monitor displacement of the tissue surface resulting from the force.

### BRIEF DESCRIPTION OF THE DRAWINGS

FIG. 1 illustrates a system for detecting tissue surface properties according to an embodiment of the present invention.

FIG. 2 is a schematic view of the system for detecting tissue surface properties illustrated in FIG. 1.

FIG. 3 is a schematic view of a device in the system illustrated in FIGS. 1-2.

FIG. 4 is a schematic view of a device in the system illustrated in FIGS. 1-3.

FIG. 5 is a schematic view of a device in the system illustrated in FIGS. 1-4.

FIG. 6 is a schematic view of a device in the system illustrated in FIGS. 1-5.

FIG. 7 is a schematic view of the system in operation.

FIG. 8 is a schematic diagram of a test platform of the system used in a study.

FIG. 9 is a perspective view of the test platform illustrated in FIG. 8.

FIG. 10 is a schematic view and image of a device in the system illustrated in FIGS. 1-5.

FIG. 11 is a graphical representation of tissue indentation depth plotted as a function of d_{R} as reported in the study.

FIG. 12 is a graphical representation of tissue indentation depth error Δδ as a function of d as reported in the study.

FIG. 13 is a graphical representation of experimental data as reported in the study.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

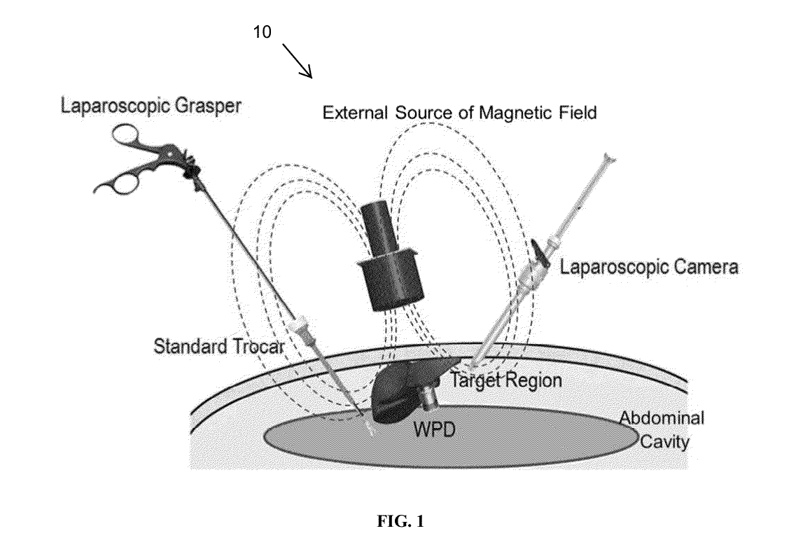

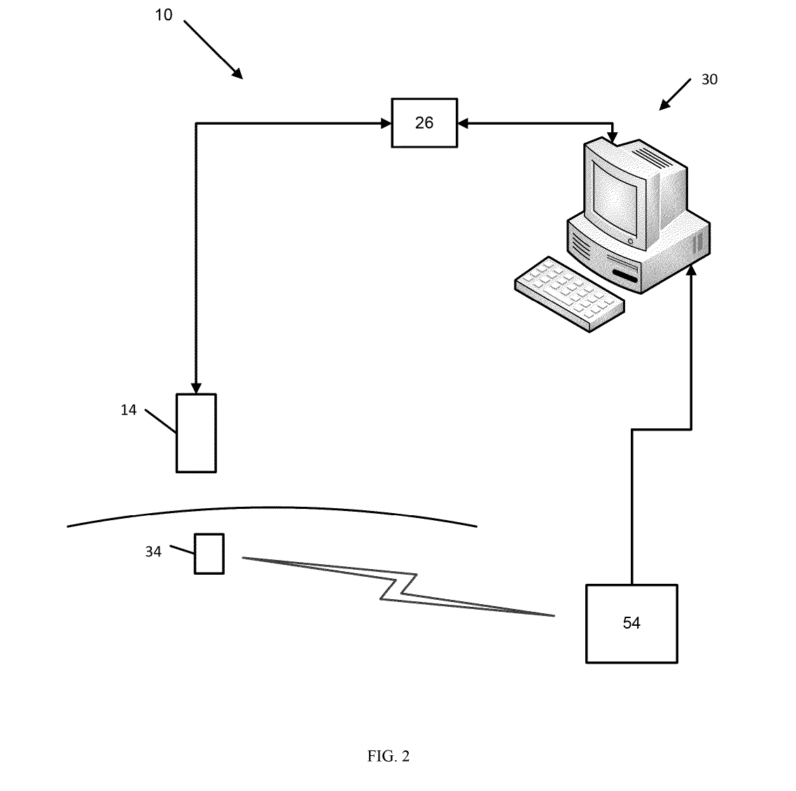

FIGS. 1-2 illustrate a system 10 for detecting tissue surface properties according to an embodiment of the present invention. For example, the system 10 can determine a tissue surface property related to local mechanical stiffness. For small indentation depths (10% of tissue thickness) it can be assumed that the tissue is linear elastic and represent the local tissue stiffness as a function of tissue reaction force and indentation depth. Since cancer tissue is stiffer than healthy tissue, the system 10 generates a stiffness map that indicates to the surgeon the location of the tumor (e.g., a region that is stiffer than the surroundings).

The system 10 includes a first device 12 having a first housing 14 positioned outside of a patient's body. The first housing 14 supports a magnetic field source 18 and a sensor 22 (e.g., force sensor). The sensor 22 can be positioned at an end of the first housing 14 that would contact the tissue. The sensor 22 can be in communication (via hardwire connection or wirelessly) with a computer program 26 configured to receive signals representing magnitude of force data from the force sensor 22. The computer program 26 when operated by a computer or processor 30 can process or compute a relevant output for presentation on a display or computer monitor. The output can represent a local stiffness map that would identify stiffer regions on the organ surface.

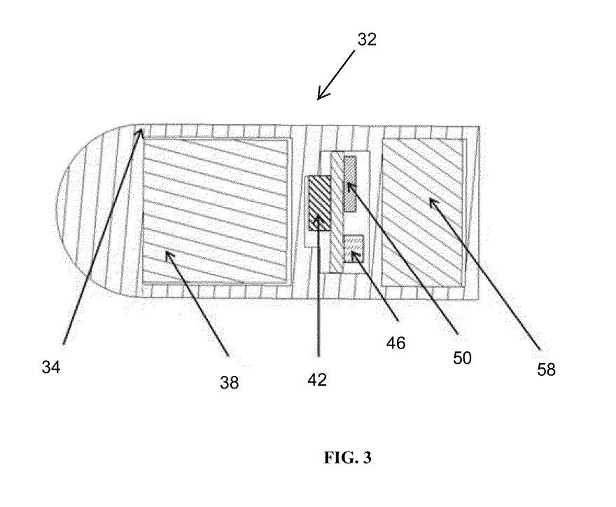

The system 10 also includes a second device 32 having a housing 34 configured for positioning inside the patient's body near a target location. With additional reference to FIG. 3, the second housing 34 supports a magnetic field source 38 (e.g., a permanent magnet), a processor or microcontroller 42, one or more sensors 46 (e.g., magnetic field sensor, a magnetometer, inertial sensor, contact sensor), a wireless telemetry unit 50 (e.g., a wireless transceiver) configured to transmit signals representing compression displacement data from the processor 42 to a receiver 54, and a power source 58 (e.g., a rechargeable battery). The receiver 54 is capable of receiving the signals from the wireless telemetry unit 50, and can further communicate the signals to the computer 30 for input to the computer program 26 (or other computer program), additional processing by the computer, and/or presentation on a display or computer monitor. For example, the output presented can be a stiffness topographical map.

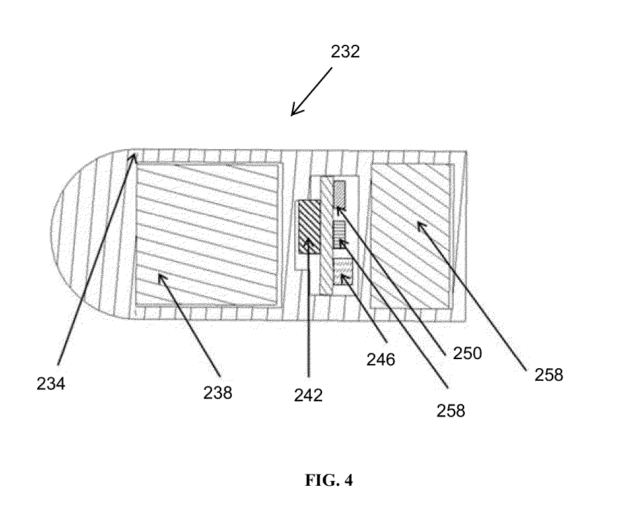

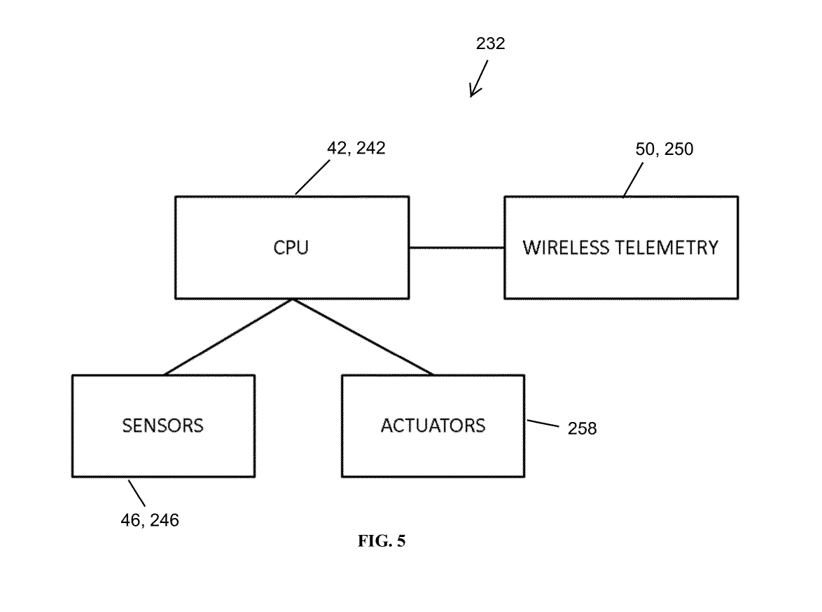

FIGS. 4-5 illustrate an alternate construction of the second device 32 (referred to as 232). The second housing 234 includes a magnetic field source 238 (e.g., a permanent magnet), a processor or microcontroller 242, one or more sensors 246 (e.g., magnetic field sensor, a Hall Effect transducer), a wireless telemetry unit 250 (e.g., a wireless transceiver) configured to transmit signals representing force data from inside the patient to the first housing 14, a power source 258 (e.g., a rechargeable battery), and one or more actuators 258 (e.g., a DC motor) configured to produce different kind of stresses on the tissue combining the attractive force between the two magnetic sources with other forces.

FIG. 5 schematically illustrates the components supported by the second housing 234. The one or more sensors 246 and the one or more actuators 258 are in communication with the processor 242 and provide signals representing force data to the processor 242. The processor 242, which is in communication with the wireless telemetry unit 250, transmits the data from the sensors 246 and actuators 258 to the wireless telemetry unit 250 for output to a receiver 54 capable of receiving the data. As noted above, the receiver 54 is capable of receiving data from the wireless telemetry unit 50, and can further communicate the data to the computer 30 for input to the computer program 26 (or other computer program), additional processing by the computer, and/or presentation on a display or computer monitor.

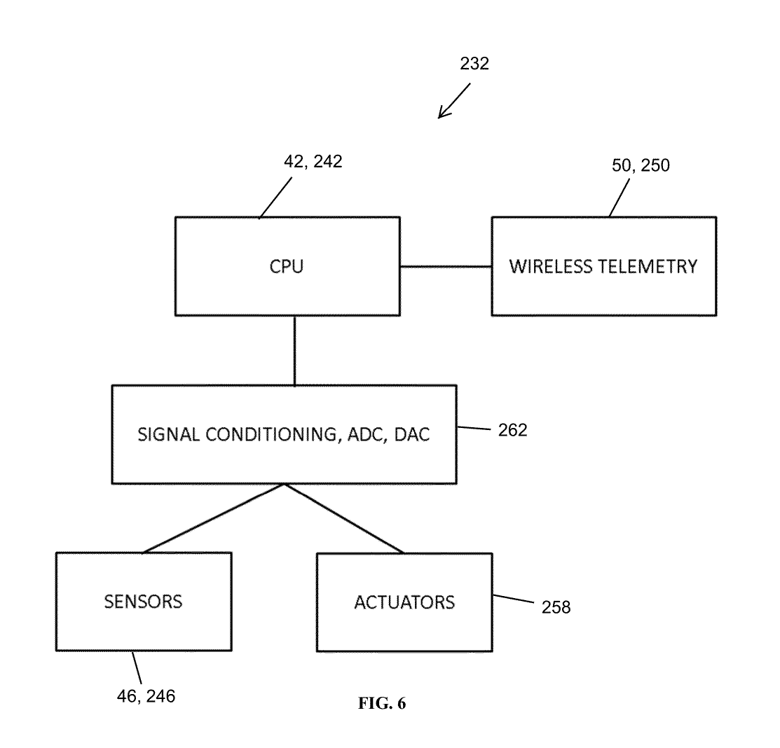

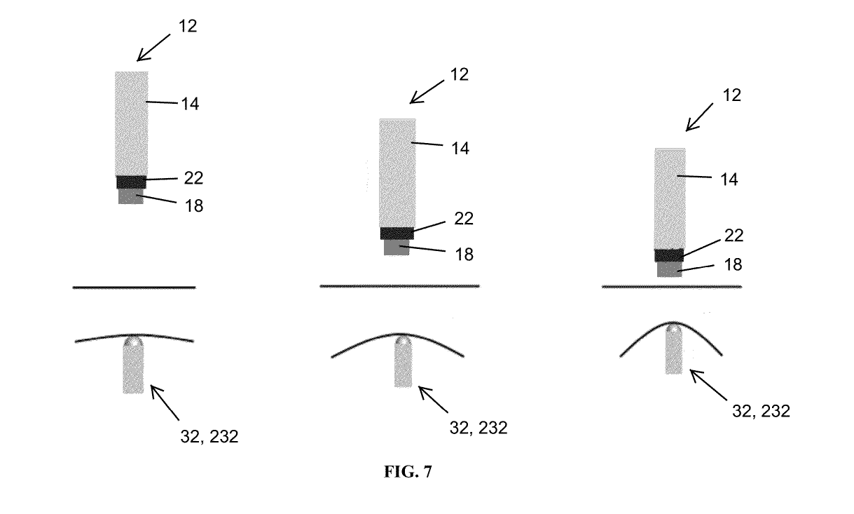

FIG. 6 illustrates an additional component 262 that may be employed in the second housing 234. The additional component 262 includes a signal conditioning unit such as an analog-to-digital converter or a digital-to-analog converter. In this construction, the signal conditioning unit 262 receives the signals from the sensors 246 and actuators 258 and conditions those signals before transmitting them to the processor 242. With reference to FIGS. 7A-C, the system 10 operates to magnetically couple the magnetic field source 18 in the first housing 14 with the magnetic field source 38 of the second housing 34. The resulting attractive magnetic force between the two magnetic sources 18, 38 can be perceived by the force sensor 22 in the first housing 14. By varying the distance between the first housing 14 and the second housing 34, the attractive force triggers a variable stress up on the tissue surrounding the second housing 34 in the direction of the external magnetic field source 18. As illustrated in FIGS. 7A-C, as the user moves the first housing 14 closer to the housing 34, the tissue experiences a different amount of stress. The resulting stress on the tissue under test is a function of the distance between the two magnetic sources, i.e., the closer the first housing 14 is with respect to the second housing 34 the greater the resulting compression stress is on the tissue. In addition, the one or more sensors 46 in the second housing 34 detect a compression displacement of the tissue due to the magnetic force variation. Because of the wireless telemetry unit 50 (or 250) embedded in the second housing 34 (or 234), the acquired data can be transmitted in real time remotely, and therefore, tissue properties can be determined.

As noted above, MIS has become popular due to the benefits of patient recovery time, less pain, and less scarring. Robotic MIS also suffers from the drawbacks discussed above since haptic feedback is not available to the surgeon because robotic surgical instruments are teleoperated from a remote console. Therefore in both MIS and robotic MIS, the surgeon is not able to leverage tactile and kinesthetic sensations to prevent accidental tissue damage or to explore tissue and organ features by palpation.

Prior research toward restoring tactile and kinesthetic sensations in MIS has focused on the distal sensing element or on the proximal rendering of haptic cues, always requiring a dedicated insertion port for the instrument. But, because surgeons do not appear willing to devote one surgical port to an instrument whose only purpose is to palpate tissues, a commercially viable solution has not been implemented. The inventors have found that having a tissue indenter (for measuring indentation pressure of the tissue using a pressure sensor) that does not take up port space may overcome this potential barrier. The inventors proposed solution to this challenge is the system 10 described above.

The inventors carried out a pilot study to assess the feasibility of wireless tissue palpation, where a magnetic device is deployed through a standard surgical trocar and operated to perform tissue palpation without requiring a dedicated entry port. The pilot study is described below.

The proposed platform used in the pilot study is composed of a wireless palpation device and a robotic manipulator holding a load cell and a permanent magnet. The wireless device included a sensing module, a wireless microcontroller, a battery, and a permanent magnet housed in a cylindrical shell (about 12.7 mm in diameter and about 27.5 mm in height). This preliminary study assessed the precision in reconstructing the indentation depth leveraging on magnetic field measurements at the wireless device (i.e., 0.1 mm accuracy), and demonstrated the effectiveness of wireless vertical indentation in detecting the elastic modulus of three different silicone tissue simulators (elastic modulus ranging from 50 kPa to 93 kPa), showing a maximum relative error below 3%. Finally, wireless palpation was used to identify differences in tissue stiffness due to a spherical lump embedded into a porcine liver. The reported results have the potential to open a new research stream in the field of palpation devices, where direct physical connection across the abdominal wall is no longer required.

Materials

A. Principle of Operation

With reference to FIG. 1, an external source of magnetic field and a wireless palpation device (WPD), which included a miniature permanent magnet and wireless electronics. The WPD was introduced into the abdominal cavity through a standard trocar and positioned on the target by a laparoscopic grasper. Then, tissue indentation was obtained by properly modulating the gradient of the external magnetic field. In order to generate kinesthetic data, the indentation depth and the pressure applied on the tissue must be known at any given time. In this pilot study, the inventors restricted the investigation to a single degree of freedom (i.e., vertical indentation) as a first step toward proving the feasibility of the proposed approach.

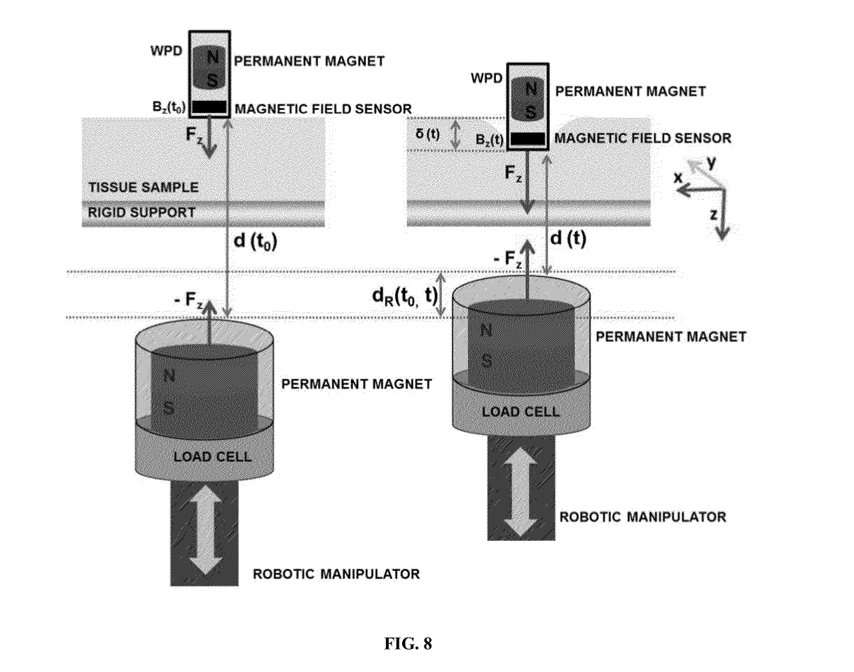

A permanent magnet mounted at the end effector of a robotic manipulator was adopted as an external source of magnetic field. Considering the two magnets (i.e., the one inside the WPD and the one at the external manipulator) oriented as in FIG. 8, the inventors studied the indentation of a tissue sample along the vertical direction by cyclically translating the external magnet along the Z axis. Neglecting gravity and assuming a pure vertical motion for the WPD, the pressure exerted on the tissue was provided by the ratio of the intermagnetic force along the Z axis, F_{z}, and the area of the WPD face in contact with the tissue. At equilibrium, the intensity of F_{z} was measured by placing a load cell in between the external permanent magnet and the end effector of the manipulator. For vertical indentation as represented in FIG. 8, gravity force acting on the WPD was considered as a preload on the tissue and factored out as an offset in the indentation trial. For any other configuration, an accelerometer can be embedded in the WPD to provide the inclination, thus allowing quantification of the exact contribution of the gravity force, should this vary during indentation. In this study, the inertial sensor was mainly used to verify the assumption of pure vertical motion for the WPD. The indentation depth δ(t) was evaluated by measuring the Z component of the magnetic field at the WPD. In particular, referring to FIG. 8 and focusing on the tissue loading phase, it is possible to express the distance between the external magnet and the internal magnet at the generic instant t as:

d(t)=d(t₀)−δ(t)−d_{R}(t₀,t) Eq. 1

where d_{R}(t₀,t) is the vertical distance traveled by the robotic manipulator since the beginning of the loading phase occurred in t₀. Since the motion of the external magnet is limited to the Z axis and the WPD is aligned on that same direction in virtue of magnetic coupling, we assumed that the Z component of the magnetic field at the WPD, B_{Z}(t), is an univocal function of d(t):

B_{Z}(t)=Φ[d(t)] Eq. 2

that can be numerically quantified through experimental calibration. Therefore, the indentation depth δ(t) can be expressed by merging Eq. 2 with Eq. 1 and rearranging the terms as:

δ(t)=Φ[B_{Z}(t₀)]⁻¹−Φ[B_{Z}(t)]⁻¹−d_{R}(t₀,t) Eq. 3

Since the value of d_{R}(t₀,t) is available at any given time from the manipulator encoders and B_{Z}(t) can be measured by placing a Hall effect sensor in the WPD, the total indentation depth can be computed at any given time during the loading phase. Same mathematical formulation applies—mutatis mutandis—to the tissue unloading phase.

A relevant assumption for the proposed approach consists in considering all the tissue deformation occurring at the interface with the WPD. This holds true for the schematization represented in FIG. 8—where the tissue under test is laying on a rigid support. However, it may not be valid as well in in vivo conditions, where the organ may lay on a softer tissue. This approximation is well accepted in the field of in vivo tissue indentation, as long as the indentation depth is relatively smaller (at least 10%) than the thickness of the organ under test.

B. Experimental Platform Overview

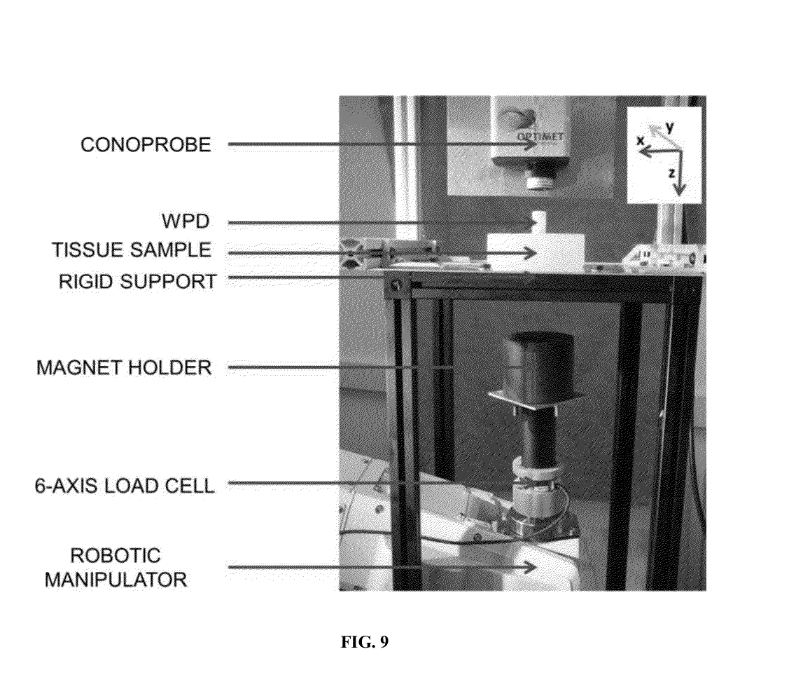

The experimental platform used to assess wireless tissue palpation for a single degree of freedom is represented in FIG. 9. It included the WPD, the robotic manipulator, and the tissue sample under test.

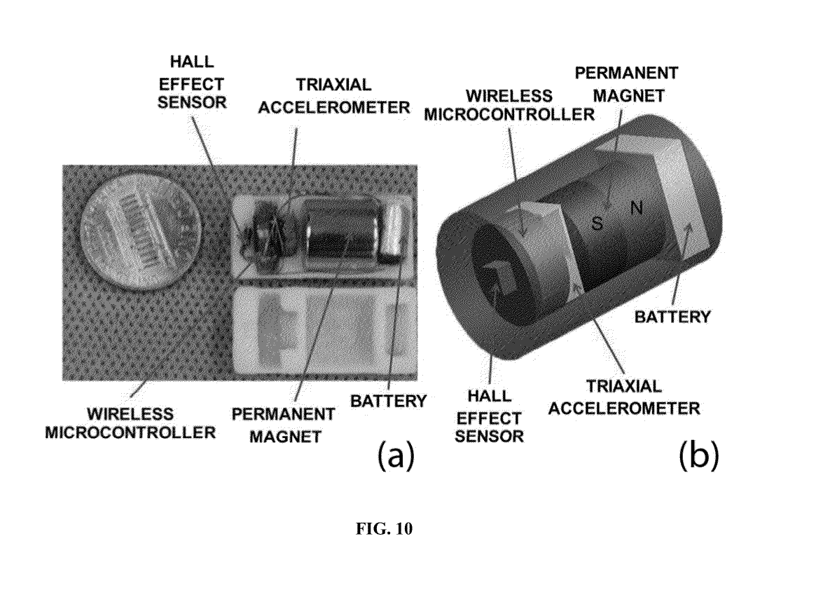

The WPD embedded a permanent magnet, a sensing module, a wireless microcontroller, and a battery into a cylindrical shell (FIG. 10). We selected an off-the-shelf cylindrical NdFeB permanent magnet (K&J Magnetics, Inc., USA), 11 mm in diameter and 11 mm in height, with N52 axial magnetization (magnetic remanence of 1.48 T). The sensing module included a Hall effect sensor (CYP15A, ChenYang Technologies GmbH & Co. KG, Germany) to measure B_{Z}, and a triaxial accelerometer (LIS331AL, STMicroelectronics, Switzerland)—to verify that the WPD motion during indentation was limited to the Z direction.

An analog signal conditioning stage connected to the Hall effect sensor output allowed to cancel out the offset due to the onboard permanent magnet (i.e., 120 mT), to apply a low-pass filter (cut-off frequency of 30 Hz), and to amplify by 29 the magnetic field signal, resulting in a resolution of 0.32 mT and a sensing range of ±130 mT. An analog to digital converter (ADC) (ADS8320, Texas Instrument, USA) was used to acquire this voltage with a sampling rate of 1 kHz and a resolution of 16 bits. The result of the conversion was then transmitted through a serial synchronous interface to the wireless microcontroller (CC2530, Texas Instruments, USA). The signals generated by the accelerometer—that did not require a 16-bit resolution—were acquired directly by the microcontroller through its embedded 12-bit ADC at 100 Hz. Accuracy after digitalization resulted in 0.35 mT for the Hall effect sensor and 1.4 degrees for the accelerometer used as inclinometer. Real-time clock timestamps were associated with each single measurement to enable synchronization with signals acquired by the external platform. The data were transmitted over a 2.4 GHz carrier frequency to a receiving unit located in the same room and connected to a personal computer, where data were elaborated, displayed, and stored. The use of a 2.4 GHz carrier frequency was previously demonstrated to be effective in transmitting data through living tissues. The wireless microcontroller was integrated in a custom-made 9.8 mm diameter printed circuit board, together with radiofrequency components. A digital switch driven by the microcontroller was placed between the battery and the sensing circuitry, in order to save battery power when measurements are not required.

A 15 mAh, 3.7 V rechargeable LiPo battery (030815, Shenzhen Hondark Electronics Co., Ltd., China) was used as the power supply. The battery layout (8 mm×15 mm×3 mm) was reduced to fit the cylindrical shell. Considering that data acquisition and transmission requires an average of 33 mA, battery lifetime was almost 30 minutes. Operational lifetime can easily be extended to fit application requirements by maintaining the WPD in sleep mode (average current consumption of 1.5 μA) and waking up the system by remote triggering whenever a palpation task is going to be performed.

As represented in FIG. 10, all the components were integrated inside a cylindrical plastic shell fabricated by rapid prototyping (OBJECT 30, Object Geometries Ltd, USA). Due to its small size (12.7 mm in diameter and 27.5 mm in height), the WPD was introduced through a 12-mm surgical trocar (e.g., the 5-12 Vesaport Plus, Covidien, USA; has an inner diameter of 13 mm). An axial-symmetric design was pursued in order to keep the WPD center of mass along its main axis, thus guaranteeing a uniform pressure on the tissue. Considering vertical indentation, the WPD surface in contact with the tissue was 113 mm², while the total weight was 10 g. It is worth mentioning that a tether can be connected to the WPD, should the surgeon feel the need for a fast retrieval of the palpation device in case of failure.

Concerning the external part of the platform—represented in FIG. 9—an off-the-shelf cylindrical NdFeB permanent magnet (5 cm in diameter and 5 cm in height), with N52 axial magnetization (magnetic remanence of 1.48 T), was adopted. Considering an average thickness of the abdominal wall upon insufflation of 30 mm, this magnet was selected on the basis of numerical analysis to operate at a distance along Z ranging from 35 mm to 75 mm away from the WPD. In this region, the simulated values of the field gradient range from 3.75 T/m to 0.6 T/m, respectively. Considering the features of the magnet embedded in the WPD, the expected intermagnetic force spans from 4.7 N to 0.75 N.

Should the required working distance be increased due to specific patient constraints (e.g., larger body mass index), an external magnet with different features can be selected by running numerical simulations again.

The magnet was embedded in a plastic holder connected to a 6-axis load cell (MINI45, Ati Industrial Automation, Inc., USA), having a resolution of 65 mN for the Z component of the force. The magnet-load cell assembly was mounted at the end effector of a six degrees of freedom industrial robot (RV6SDL, Mitsubishi Corp., Japan), presenting a motion resolution of 10 μm along the Z direction. It is worth mentioning that the holder was designed to space the magnet enough from the load cell and the manipulator to prevent electromagnetic interferences. Data from the load cell were acquired by a dedicated acquisition board (NI-PCI 6224, National Instruments, USA) at a sampling frequency of 1 kHz, and merged with the manipulator position and the signals coming from the WPD.

A 34 mm thick tissue sample—silicone (M-F Liquid Plastic, MF Manufacturing, USA) in different stiffnesses or porcine liver, depending on the trial—was placed on a 2 mm thick rigid support, as represented in FIG. 9.

Finally, the algorithm described by Eq. 3 was implemented in Matlab (Mathworks, USA) upon experimental calibration.

In particular, the numerical function Φ⁻¹ was evaluated by placing the WPD directly on the rigid support and by recording B_{Z}(t) while moving the external magnet at a constant speed (i.e., 3.12 mm/s) from a starting position 75 mm away from the rigid support along the Z axis (i.e., d_{R} varying from 0 mm to 75 mm, where for d_{R}=75 mm the top part of the holder was almost in contact with the lower side of the rigid support). This measurement was performed for five loading-unloading cycles, and the values were averaged. Given the exponential decay of the magnetic field with distance, a fifth-order polynomial function was used to fit Φ⁻¹, thus obtaining:

$$\begin{matrix}d ( t ) = {Φ}^{- 1} [ {B}_{Z} ( t ) ] \\ = {\sum }_{i = 0}^{5} {a}_{i} \cdot {{B}_{Z} ( t )}^{i}\end{matrix} \qquad Eq . 4$$

with a₀=185.6 mm, a₁=−6.95·10³ mm/T, a₂=1.57·10⁴ mm/T², a₃=−2·10⁷ mm/T³, a₄=1.31·10⁷ mm/T⁴, a₅=−3.51·10⁷ mm/T⁵. The square of the correlation coefficient for the proposed fitting was R²=0.99998.

Since the polynomial function is applied to a sensor reading affected by a given uncertainty ΔB_{Z}, it is interesting to study the error propagation to the indentation depth δ. Considering δ(t) as expressed in Eq. 3, we can write its absolute error as a function of ΔB_{Z} and Δd_{R}:

$$\vert Δδ \vert = \vert \frac{\partial {Φ}^{- 1} [ {B}_{Z} ( t ) ]}{\partial {B}_{Z} ( t )} \vert \cdot \vert Δ {B}_{Z} \vert + \vert Δ {d}_{R} \vert \qquad Eq . 5$$

Considering Eq.3 and a negligible error in d_{R}—reasonable assumption given the high resolution of motion for the manipulator, we then have

$$\vert Δδ \vert = \vert {\sum }_{i = 1}^{5} i \cdot {a}_{i} \cdot {{B}_{Z} ( t )}^{i - 1} \vert \cdot \vert Δ {B}_{Z} \vert \qquad Eq . 6$$

This equation clearly shows how the accuracy of the proposed method depends upon the strength of the magnetic field at the WPD, which for the proposed platform, is a function of the distance between the external magnet and the WPD.

Experimental Results

Experimental validation of single degree of freedom wireless palpation consisted in three different trials. First, the effectiveness of the algorithm in reconstructing the indentation depth from magnetic field values was assessed. Then, three silicone tissue simulators, each with a different elastic modulus, were indented with the proposed approach, and the results compared with standard indentation. Finally, a spherical lump was embedded in a porcine liver and wireless palpation was used to identify differences in tissue stiffness.

A. Indentation Algorithm Assessment

An optical conoscopic holography sensor (Conoprobe, Optimet, USA) was adopted as reference measurement system. The conoprobe was mounted so to point the laser spot on the upper circular surface of the WPD, as in FIG. 9. The indentation test was performed on a squared silicone tissue sample (elastic modulus 6.45 kPa, thickness 34 mm, lateral side 74 mm) for d_{R} varying at a constant speed (i.e., 3.12 mm/s) from 0 mm to 41 mm, where for d_{R}=41 mm the top part of the holder was almost in contact with the lower side of the rigid support. Five loading-unloading trials were carried out and error analysis was performed on the acquired data. Accelerometer output confirmed that WPD motion was always occurring along the Z direction.

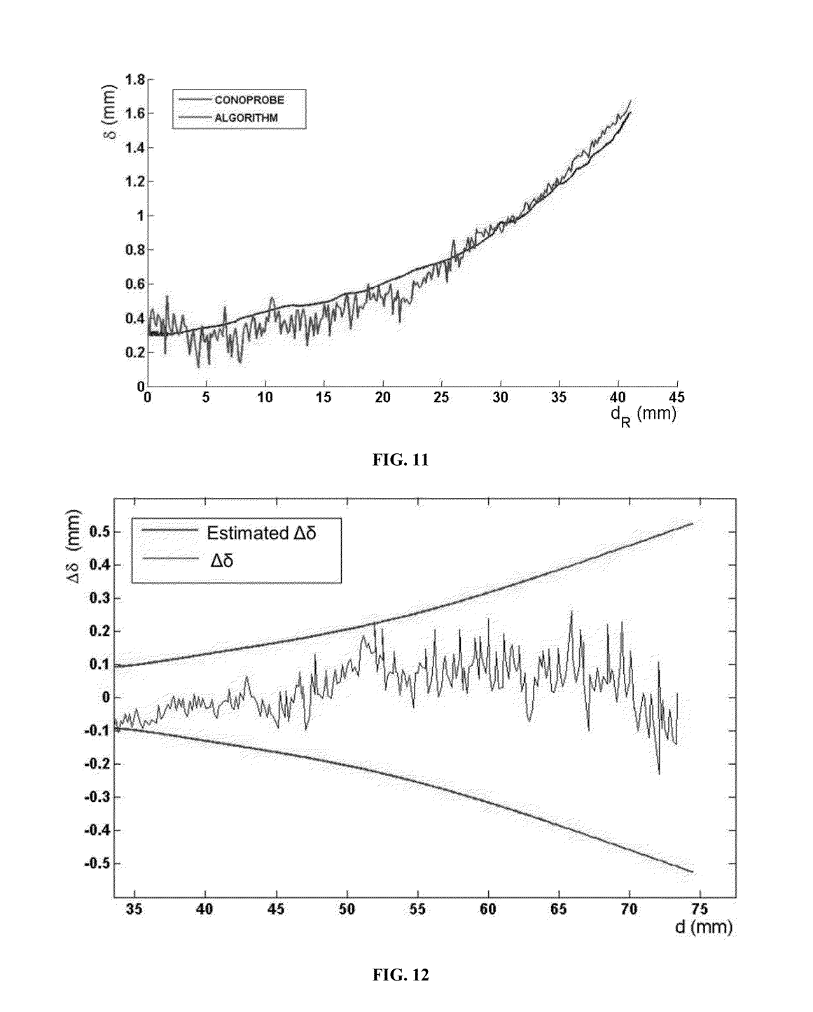

A typical loading plot for δ(t) acquired with both the reference system and the proposed approach is represented in FIG. 11 as a function of d_{R}. Considering the tissue sample thickness, the rigid support, and the recorded indentation depth, the distance d from the external magnet to the WPD varied from 75 mm to 35 mm during the trials.

Concerning the error, the Hall effect sensor measurements presented a standard deviation of ±0.3 mT. By using this value in Eq. 6 as ΔB_{Z}, it is possible to plot an envelope of the expected standard deviation of the tissue indentation depth δ as a function of the distance d (FIG. 12). For all the acquired measurements, the difference between the conoprobe reading and the reconstructed δ always fell within the envelope. One example is given in FIG. 12. From the same plot it is possible to see that the standard deviation for δ is ±0.1 mm at 35 mm, while increases to ±0.5 mm at 75 mm.

B. In Vitro Trials

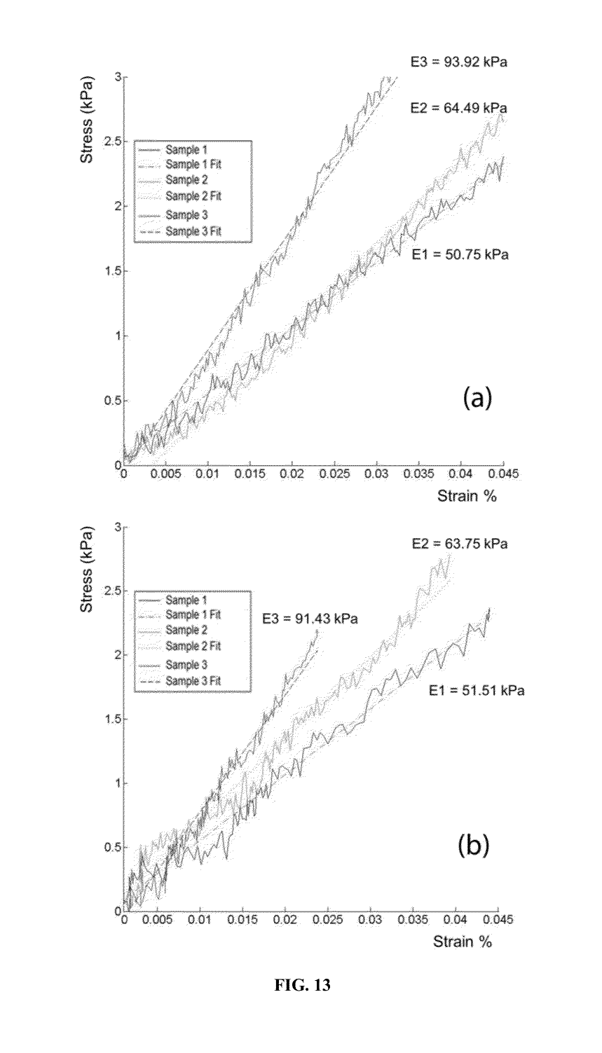

In order to validate the effectiveness of wireless palpation to detect the elastic modulus of a tissue sample as a traditional indenter, three squared silicone tissue simulators (thickness 34 mm, lateral side 74 mm) were fabricated, each with a different proportion of hardener (i.e., 20%, 25%, and 30%), thus resulting in different elastic moduli E1, E2, and E3. A traditional vertical indenter was obtained by replacing the magnet holder with a cylindrical probe at the interface with the load cell. The probe was designed to have the same contact area as the WPD. The indenter probe was first driven to touch the surface of the tissue layer with a preload of 0.2 N. Five loading-unloading trials—reaching an indentation depth of 3 mm—were performed for each tissue sample at a constant speed of 3.12 mm/s. Stress-strain plots obtained from a single loading are represented in FIG. 13(a). Measured elastic moduli were E1=50.75 kPa, E2=64.49 kPa, and E3=93.92 kPa.

Wireless palpation was then performed on the same three samples. Five loading-unloading trials were performed by following the same protocol described for the assessment of the indentation algorithm described above. The results are reported in FIG. 13(b). Also in this case, accelerometer data confirmed that WPD motion was always occurring along the Z direction. Indentation force reached 2.2 N, while maximum indentation depth was 2.4 mm for the softer sample.

Considering all the performed trials, the average relative error for wireless palpation in measuring the elastic modulus was 1.49%, 1.14% and 2.65% for the tissue samples having E1, E2, and E3, respectively.

C. Ex Vivo Trials

A freshly excised porcine liver was used for the ex vivo trials. A 5 mm diameter sphere, fabricated by rapid prototyping in hard material, was embedded close to the tissue surface so as to simulate a hidden malignant liver tumor that is usually stiffer than the surrounding healthy tissue.

While most of our research work has focused either on providing force and tactile sensing at the end effector, or enabling haptic rendering at the user interface, the proposed approach tackles the physical connection between the two sides of the palpation instrument. The reported results lead to the conclusion that wireless vertical indentation is feasible in a laboratory setting, showing comparable results to traditional indentation techniques.

Various features and advantages of the invention are set forth in the following claims.

What is claimed is:

1. A system for detecting a tissue property, the system including:
a first unit located on a first side of the tissue surface, the first unit including
a first housing,
a first sensor supported by the first housing, and
a first magnetic field source supported by the housing; and
a second unit located on a second side of the tissue surface, the second unit including
a second housing,
a second sensor supported by the second housing,
a second magnetic field source supported by the second housing,
a controller,
a telemetry unit, and
a power source;
wherein the first unit and second unit are magnetically coupled such that a force created therebetween generates a first stress on the tissue surface;
wherein the first sensor is configured to sense a magnitude of the force;
wherein the second sensor is configured to determine a displacement of the tissue due to the first stress.

2. The system of claim 1 further comprising an actuator supported by the second housing.

3. The system of claim 2 wherein the actuator provides a second stress on the tissue surface, and wherein the second sensor is configured to determine a displacement of the tissue due to the first stress and the second stress.

4. The system of claim 1 further comprising a computer system in communication with the telemetry unit, and wherein the computer system is configured to receive signals transmitted by the telemetry unit.

5. The system of claim 4 wherein the telemetry unit is configured to receive signals from the second sensor and to transmit those signals from the second sensor to the computer system.

6. The system of claim 5 wherein the computer system further comprises a processor and a software program configured to process the signals from the telemetry unit and present data on a display related to tissue displacement.

7. The system of claim 6 wherein the first sensor is in communication with the computer system, and wherein the software program is configured to process the signals from the first sensor and present data on the display related to magnitude of force and tissue displacement.

8. The system of claim 1 wherein the second unit further includes a signal conditioner configured to condition the signals transmitted from the second sensor to the controller.

9. A system for detecting a tissue property, the system including:
a first unit positioned exterior to a patient, the first unit including
a first housing,
a first sensor supported by the first housing, and
a first magnetic field source supported by the housing; and
a second unit positioned inside of a patient near a target tissue, the second unit including
a second housing,
a second sensor supported by the second housing, the second sensor positioned adjacent the target tissue,
a second magnetic field source supported by the second housing,
a controller supported by the second housing,
a telemetry unit supported by the second housing, and
a power source supported by the second housing;
wherein the first sensor is configured to detect an amount of force applied to the target tissue due to a magnetic coupling between the first unit and the second unit;
wherein the second sensor is configured to determine a displacement of the tissue due to the magnetic coupling between the first unit and the second unit.

10. The system of claim 9 further comprising an actuator supported by the second housing.

11. The system of claim 10 wherein the actuator provides a stress on the target tissue, and wherein the second sensor is configured to determine a displacement of the tissue due to the magnetic coupling between the first unit and the second unit.

12. The system of claim 9 further comprising a computer system in communication with the telemetry unit, and wherein the computer system is configured to receive signals transmitted by the telemetry unit.

13. The system of claim 12 wherein the telemetry unit is configured to receive signals from the second sensor and to transmit those signals from the second sensor to the computer system.

14. The system of claim 13 wherein the computer system further comprises a processor and a software program configured to process the signals from the telemetry unit and present data on a display related to tissue displacement.

15. The system of claim 14 wherein the first sensor is in communication with the computer system, and wherein the software program is configured to process the signals from the first sensor and present data on the display related to magnitude of force and tissue displacement.

16. The system of claim 9 wherein the second unit further includes a signal conditioner configured to condition the signals transmitted from the second sensor to the controller.

17. A method for detecting a tissue property, the method including:
positioning a first unit on a first side of the tissue surface at a region of interest, the first unit including a first housing that supports a first sensor, a first magnetic field source, a controller, a telemetry unit, and a power source;
providing a second unit on a second side of the tissue surface, the second unit including a second housing having a second sensor and a second magnetic field source;
modulating a force created by a magnetic field between the first and second units; and
determining and monitoring displacement of the tissue surface resulting from the force.

18. The method of claim 17 further comprising transmitting data related to the displacement of the tissue surface from the telemetry unit to a computer system and displaying the data.

19. The method of claim 18 further comprising transmitting data related to magnitude of force from the second sensor to the computer system and displaying the data.

20. The method of claim 17 wherein the magnetic field is generated between the first magnetic field source and the second magnetic field source.
